(19) United States
(12) Patent Application Publication
(10) Pub. No.: US 2014/0348890 A1
(43) Pub. Date: Nov. 27, 2014

(54) WOUND DRESSING

(71) Applicant: MARIUS-THOMAS GORKA, MULHEIM-KARLICH (DE)

(72) Inventor: MARIUS-THOMAS GORKA, MULHEIM-KARLICH (DE)

(73) Assignee: LOHMANN & RAUSCHER GMBH & CO. KG, NEUWlED (DE)

(21) Appl. No.: 14/455,468

(22) Filed: Aug. 8, 2014

Related U.S. Application Data
(63) Continuation of application No. 13/473,103, filed on May 16, 2012.

(30) Foreign Application Priority Data
May 19, 2011 (EP) 11 004 166.2

(51) Int. Cl. A61L 15/44; A61L 15/22; A61L 15/26

(52) U.S. Cl. CPC A61L 15/44; A61L 15/26; A61L 15/225; A61L 2300/206; A61L 2300/404
USPC 424/404; 424/445; 424/78.07

(57) ABSTRACT

The invention relates to a wound dressing having a backing and an antimicrobial wound contact layer, wherein the wound contact layer has a hydrophobized active ingredient.

### CROSS-REFERENCE TO RELATED APPLICATIONS

This application claims priority to and is a continuation of U.S. patent application Ser. No. 13/473,103, filed on May 16, 2012, which claims priority to European Patent Application No. 11 004 166.2, filed on May 19, 2011, each of which is incorporated herein by reference in its entirety.

### STATEMENT REGARDING FEDERALLY SPONSORED RESEARCH OR DEVELOPMENT

Not applicable.

### BACKGROUND OF THE INVENTION

The invention relates to a wound dressing having a backing and an antimicrobial wound contact layer as well as a material for producing a wound contact layer for a wound dressing.

Antiseptic wound dressings are used for topical treatment of traumatic wounds such as cut wounds and scrapes as well as chronic wounds such as decubital ulcers, leg ulcers, and the like, burns and postoperative wounds. An antimicrobial finish imparts a local antimicrobial activity to the wound dressing. The wound dressing finished in this way may be applied to infected and critically colonized wounds as well as wounds at risk of infection in both humans and animals.

In view of growing requirements of hygienic standards in the medical and clinical fields, there is an increasing demand for antiseptic wound dressings, bandaging materials and other materials. At the same time, high demands must be made of the antimicrobial activity and bioavailability of such materials.

Known antibiotics and/or broadband antibiotics as well as antimicrobial substances such as triclosan or silver in any form are problematical with regard to the antimicrobial activity because resistances of pathogenic microorganisms to these active ingredients have developed, so the antimicrobial activity no longer exists to the desired extent.

Since the 1990s polyhexanide (polyhexamethylene biguanide, PHMB), a very popular disinfectant with antibacterial, antimycotic, antiviral and antiprotozoic activities, has also been used to an increasing extent in therapeutic applications, e.g., in surgery, ophthalmology and for wound treatment. PHMB is a polycation whose antibacterial activity is based on the interaction with negatively charged phospholipids of bacterial cell walls. PHMB has a very broad-spectrum of effect as an antiseptic and is also effective against yeasts and methicillin-resistant Staphylococcus aureus (MRSA) for example. At the same time, the tissue tolerability of PHMB can be classified as very good because the neutral lipids of human cells hardly enter into any interaction with PHMB. Furthermore PHMB is very biocompatible in comparison with other antiseptics (cf. Polyhexanide: A safe and highly effective biocide, K. Kaehn, Table 3, Toxicological Data of Known Antiseptics in Comparison with PHMB). In addition, it should be pointed out that currently there are no known resistances to PHMB.

PHMB is used as a broad-spectrum bactericide in body care applications (DE 698 17 654 T2), industrial applications and medical products and is commercially available as an aqueous solution in the form of a hydrochloride salt (Cosmocil CQ, Cosmocil FQ, Arch). For most applications, the use of PHMB is very suitable as a hydrophilic hydrochloride salt that is readily soluble in water. Similar behavior is also exhibited by other antiseptics such as chlorhexidine (1,1′-hexamethylenebis[5-(4-chloro-phenyl)biguanide]digluconate) and alexidine (1,1′-hexamethylenebis[5-(4-(2-ethylhexyl) [symbol] phenyl)biguanide]diacetate).

EP 1,272,229 B1 describes an antiseptic compress having a wound contact layer comprised of an elastomer matrix, an apolar oil, a hydrocolloid (sodium carboxymethyl cellulose) as a dispersion and at least one surfactant (Tween 80), optionally with at least one antimicrobial agent (silver sulfadiazine). When using corresponding compresses, the development of a resistance is observed. Furthermore, the use of surfactants is problematical because they have a certain cytotoxic potential. It may also be regarded as problematical that silver sulfadiazine requires a prescription.

EP 1 691 851 B1 describes a wound dressing having a liquid-permeable substrate which is provided with openings and having an absorbent (at least 50% of the dry weight) non-adhesive polymer composition which contains a hydrophobic organic polymer matrix, a plasticizer and hydrophilic organic microparticles plus optionally bioactive agents. The aforementioned document describes synthetic and natural bioactive agents. When using these agents, it has proven problematical that the absorbent (superabsorber) in the dressing binds the liquid in the form of a gel. Due to the swelling process, the openings for exudate (e.g., in viscous form) become almost impassable. Free removal of the exudate into the absorbent secondary dressing may then be hindered.

WO 0203899 describes a dressing impregnated with an aqueous hydrophilic PHMB variant. It has been observed that when using this dressing, the backing sticks to the wound, and it may even happen that granulation tissue grows into the dressing. Therefore, despite the good tolerability of PHMB and the lack of development of a resistance, the product described in the aforementioned document has only limited suitability for wound treatment.

The dressing can be prevented from sticking to the wound by using a sterile non-adhesive compress according to EP 1 143 895 B1. This compress contains an elastomer matrix of a three-block elastomer, an oily plasticizer, petroleum jelly and hydrophilic particles of a hydrocolloid (CMC, alginate) and optionally an active ingredient. Active ingredients proposed in the aforementioned document include silver sulfadiazine, antibiotics, for example, neomycin or polymycin and steroidal or non-steroidal anti-inflammatory drugs (NSAIDs), for example, triamcinolone acetonide. These active ingredients have only limited benefit.

### BRIEF SUMMARY OF THE INVENTION

In view of the problems in the prior art described above, the object of the present invention is to make available a biocompatible, non-adhesive (optionally slightly adhesive) wound dressing with a nonspecific but nevertheless effective antimicrobial finish for treatment of wounds that are infected, critical colonized and/or at risk of infection.

According to the invention, this object is achieved by a refinement of the known wound dressings which is characterized essentially in that the wound contact layer has a hydrophobized active ingredient.

Hydrophobizing the active ingredient makes it possible to design the wound contact layer as an organic polymer matrix. It is therefore possible to prevent adhesion of the backing to the wound and/or growth of granulation tissue into the dressing. At the same time, the hydrophobized active ingredient may be homogeneously distributed in the wound contact layer. Accordingly, the wound contact layer of an inventive wound dressing may have an elastomer matrix furnished with the hydrophobized active ingredient.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### DETAILED DESCRIPTION OF PREFERRED EMBODIMENT

In an especially preferred embodiment of the invention, the hydrophobized active ingredient has hydrophobized oligomer/polymer biguanide, in particular hydrophobized PHMB and/or hydrophobized oligomer/polymer guanide. This makes use of the finding that commercially available water-soluble salts of PHMB are not suitable for obtaining a homogeneous solution/mixture/dispersion in a hydrophobic organic elastomer polymer matrix. This is true in particular when the processing temperatures are above the boiling point of water and/or of the aqueous PHMB solution. Because of the hydrophilicity of the PHMB hydrochloride salt, the active ingredient is also unsuitable in anhydrous form (powder, particles, microparticles) for ensuring a homogeneous and phase-stable mixture in the hydrophobic elastomer polymer matrix. The melting range of PHMB-HCl particles (powder) is approximately 79 to 136° C., which corresponds approximately to the processing temperature. At these temperatures (processing temperatures of 110 to 150° C.) phase separation occurs, accompanied by formation of agglomerates and a heterogeneous distribution of the active ingredient in the polymer matrix.

By hydrophobizing the active ingredient, a homogeneous distribution of the antiseptic in the hydrophobic composition can be achieved without adding surfactants. The hydrophobizing may be accomplished by reducing the positive charge of the polycationic active ingredient (deprotonation) or by replacing the counterion/anion (chloride) with a more hydrophobic counterion/anion.

Within the scope of this invention it has been discovered that PHMB in the form of a salt of an organic acid containing 5 to 25 carbon atoms is very suitable for being incorporated homogeneously into polymer elastomer compositions because of the hydrophobicity and the lowered melting range (e.g., melting range of PHMB stearate is 71.2 to 86.7° C.). The high antimicrobial activity is retained.

The homogeneous distribution of the active ingredient in the elastomer matrix and thus in the coated product ensures a release of the active ingredient into the wound so that it is homogeneous over the area of the wound and prevents high local concentrations of the active ingredient and local cytotoxic effects associated with that. On the other hand, this also avoids concentrations of the active ingredient that are too low in certain regions of the wound dressing and that could lead to a gap in effect and possibly even to bacterial colonization of the wound dressing and thus at threat to the patient.

The wound contact layer of an inventive wound dressing preferably comprises an organic polymer matrix, a plasticizer and hydrophilic organic, and/or inorganic microparticles which form a gel on contact with aqueous solution.

The polymer matrix may contain a three-block polymer A-B-A, where the polymer matrix preferably contains no more than 3.2 parts by weight, in particular no more than 2.6 parts by weight of a block polymer, where the terminal block A may be of the polystyrene type, the central block B may be of the saturated polyolefin type, and the styrene content is between 25 and 40%. Hydrogenated polystyrene-polyethylene-polybutylene-polystyrene copolymers (SEBS, e.g., G1651, Kraton) are expediently used. Within the scope of the invention, hydrogenated polystyrene-b-poly(ethylene/propylene)-b polystyrene copolymers (SEEPS, Septon 4055, Kuraray) are preferred. Additionally or alternatively, the wound contact layer of an inventive wound dressing may contain a single phase ointment such as petroleum jelly mixed with the hydrophobized active ingredient, a hydrophobic Deutsches Arzneibuch (DAB) basic gel and/or a multiphase system (e.g., water-in-oil) and/or a hydrophobic silicone gel mixed with the hydrophobized active ingredient.

Wound dressings are usually sterilized to promote healing without the risk of microbial contamination. Wound infections have been shown to delay healing. They are caused by pathogenic microorganisms, which penetrate into the wound (optionally even through the wound dressing), replicate there and produce toxins which affect both the wound tissue and the body as a whole.

A variety of techniques are known for destroying contaminating microorganisms. In addition to sterilization by saturated steam or by dry heat, sterilization by gas (ethylene oxide, formaldehyde) or sterilization by irradiation is/are used routinely. However, none of these techniques is suitable without reservation for producing products containing a fat-based elastomer matrix. This relates in particular to products for pharmaceutical use. For these products, sterilization by saturated steam or dry heat can hardly be used because the elastomer matrix and the hydrocolloid cannot tolerate the high temperatures and/or the elevated atmospheric humidity. Sterilization by gas is regarded as problematical because of the resulting risk that residues may remain in the wound dressing. Furthermore, with this technique it is not generally possible to obtain a distribution of the sterilizing agent over the entire volume of the elastomer polymer composition. This limits the efficacy of this sterilization.

In general, the use of a sterile barrier (primary packaging) which preserves the sterility of the product in marketing until use on the patient, also prevents the use of most of the aforementioned sterilization methods because the packaging was selected to be airtight to suppress the oxidizing influence of atmospheric oxygen as well as the influence of atmospheric humidity on the hygroscopic hydrocolloid dispersed in the matrix.

For the aforementioned reasons, sterilization by irradiation is generally used. This ensures effective sterilization into the interior of the product. Two types of radiation may be used namely β- and γ-radiation.

The sterilization dose is adjusted as a function of the initial microbiological burden (bioburden), i.e., the quantity of microorganisms present on/in the product prior to sterilization, within the framework of a dose determination.

To achieve effective decontamination with an adequate safety margin, an average dose of 25 kGray is generally used for the products to be sterilized. In practice, a product receives a dose which varies between 25 and 40 kGray, depending on the method used.

However, it has been found that sterilization by irradiation has an influence on the elastomer matrix treated in this way. In particular, the energy introduced into the matrix by the radiation is high enough to break the carbon-carbon and carbon-hydrogen bonds of the elastomers used and possibly also to cause breaks in the chains in these polymer macromolecules and reductions in their average molecular weight, which would influence and/or reduce their properties in particular their cohesion ability. Consequently, these products are not completely satisfactory either because of difficulties in conjunction with handling in applying or removing the dressing or because of a relatively high polymer prices or a certain tissue intolerance of the elastomer polymers or due to the loss of cohesion after radiation sterilization.

Within the context of the present invention, it has proven to be especially advantageous if the wound contact layer to solve these problems contains an elastomer matrix comprising less than 3.2 wt % of an elastomer in particular 3.0 wt % or less, preferably less than 2.7 wt % of a polymer component, thereby yielding better tissue tolerability in addition to the economic advantage.

To achieve especially good sterilization stability, in an especially preferred embodiment of the invention, elastomers and/or elastomer mixtures with an especially high molecular weight are used to counteract the negative effects of radiation sterilization on the cohesion of the matrix. According to the invention, elastomers with a molecular weight of 200,000 to 300,000 dalton, distributed under the brand name Septon 4055, may be used as the high molecular polymers. It has surprisingly been found that adding ultrahigh molecular elastomers such as those polymers distributed under the brand name Septon 4077, with a molecular weight of 400,000 to 450,000 dalton greatly improves the cohesion after sterilization. Due to the presence of relatively temperature-sensitive components such as petroleum jelly or CMC in the matrix, the processing temperature is limited, preferably being no higher than approximately 140° C. to 145° C. However, the expected increase in processing temperature with the use of an ultrahigh molecular elastomer (Mw=approximately 400,000 to 450,000 dalton) is only minor and is within the acceptable range.

In the sense of ensuring good processability, it has proven to be especially expedient within the context of the invention if the total polymer content of the elastomer matrix comprises at least 50% of a high molecular elastomer with a molecular weight of approximately 200,000 to 300,000 dalton, the remainder being an ultrahigh molecular polymer with a molecular weight of 400,000 to 450,000 dalton.

To prevent problems in processing corresponding elastomers, it has proven advantageous if the molecular weight of the ultrahigh molecular polymer is less than 600,000 dalton, in particular less than 550,000 dalton.

The Brookfield viscosity of polymers used according to the invention is expediently at least 5000 mPas (for a 10% solution in toluene at 30° C.).

Of the products/plasticizers that are readily suitable for plasticizing the elastomer, reference may be made in particular to fatty substances that are liquid or solid at room temperature, in particular paraffin oils, medicinal white oils, mineral oils, ointment paraffins, petroleum jelly, silicone oils or silicone fats and/or waxes as well as mixtures thereof. Plasticizers such as petroleum jelly, whose drop point is between 35° C. and 70° C., are preferred. Medicinal white oils, whose purity requirements conform to Ph. Eur., are also preferred.

The hydrocolloids, which are known in general (CMC, alginates, gelatin, xanthan, pectins) but also silicates such as bentonites, aerosils or superabsorbers may be used as the hydrophilic organic and/or inorganic microparticles that bind water and undergo gelation in the process. Microparticles with a diameter of 50 to 300 μm (assuming a spherical shape), in particular with a diameter of 50 to 200 μm, are preferred.

The matrix may also contain antioxidants. Suitable antioxidants include the sulfur antioxidants, for example, the zinc dibutyl dithiocarbamate marketed by the company Akzo Nobel Chemicals under the brand name Perkacit ZDBC and/or the phenolic antioxidants, for example, the products marketed under the brand names Irganox® 1010, Irganox® 565, Irganox® 1035 by the company BASF may also be mentioned as suitable antioxidants.

The compound in Irganox® 1010 is preferred within the scope of the present invention.

The wound contact layer of an inventive wound dressing may also comprise an additive selected from the group consisting of another stabilizer, extrusion aids, fillers, pigments, dyes, crosslinking agents, odor suppressants, tackifiers, tolerability mediators and combinations therefore.

The organic acid which forms the salt of the polymer biguanide may contain a phosphonic, phosphoric, sulfonic or sulfuric acid group, but preferably contains a carboxylic acid group. The organic acid may be aromatic, but is preferably aliphatic including alicyclic. If the organic acid is aliphatic, the aliphatic chain of the organic acid may be linear or branched, saturated or unsaturated, including mixtures thereof. The aliphatic chain is preferably linear and it is also preferable for the organic acid to be an aliphatic carboxylic acid. The best hydrophobization is achieved with aliphatic carboxylic acids.

It is preferable for the organic acid to contain no less than 8, preferably no less than 10, in particular no less than 12 carbon atoms, not including the acid group. The organic acid preferably contains no more than 24, more preferably no more than 20 and in particular no more than 18 carbon atoms, not including the acid group. The organic acid may contain more than one acid group but it is preferable for only one such group to be present. If the organic acid contains more than one acid group, the effect of the hydrophobization is diminished accordingly. The organic acid may be substituted with a halogen or in particular a hydroxyl group. In the sense of the most effective hydrophobization possible, however it is proven favorable if the organic acid is free of substituents.

Some aliphatic carboxylic acids are available commercially as mixtures such as those obtained from and containing animal fats and plant oils as well as saturated and unsaturated aliphatic chains. These may also prove to be beneficial, in particular the C₁₄₋₁₈ alkylcarboxylic acids and their completely saturated or hydrogenated analogs. Examples of optionally substituted carboxylic acids include valeric acid, hexanoic acid, octanoic acid, 2-octenoic acid, lauric acid, 5-dodecenoic acid, myristic acid, pentadecanoic acid, palmitic acid, oleic acid, stearic acid, eicosanoic acid, heptadecanoic acid, palmitoleic acid, ricinoleic acid, 12-hydroxystearic acid, 16 hydroxyhexadecanoic acid, 4-hydroxydecanoic acid, dodecanedioic acid, undecaneoic acid, sebacic acid, benzoic acid, hydroxybenzoic acid and terephthalic acid.

It has proven to be especially favorable if the aliphatic carboxylic acid is stearic acid and if the polymer biguanide is PHMB. The reaction of PHMB with stearic acid to form PHMB stearate may be accomplished without any mentionable influence on the antimicrobial properties of the PHMB.

The polymer biguanide contains at least three biguanide units. The polymer biguanide preferably contains more than two biguanide units which are bound by a bridge group containing at least one methylene group. The bridge group is preferably such that there are at least three carbon atoms between the two neighboring biguanide units, but no more than 10 carbon atoms and in particular no more than 8 carbon atoms. The polymer biguanide may also be terminated by a suitable group, which may optionally be a hydrocarbon group or a substituted hydrocarbon group or an amine. The polymer biguanide used according to the invention contains at least three biguanide units, preferably between 7 and 18, especially preferably no less than 9 and no more than 17. This is preferably a linear polymer biguanide.

In the case of the preferred polyhexamethylene biguanide, it is a mixture represented by the compounds of formula 1 in the free base form in which the value of n is 4 to 40 and in particular 4 to 15. It is especially preferable for the average of n in the mixture to be 12. The average molecular weight of the polymer mixture preferably corresponds to n=10-13.

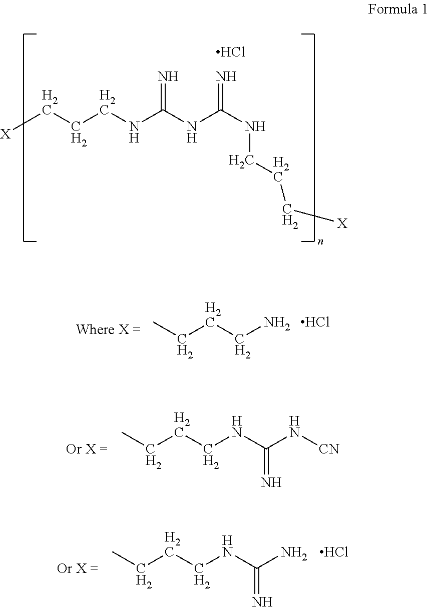

According to the present invention, a composition containing a backing mass and a polymer biguanide in the form of its salt with an organic acid having 4 to 30 carbon atoms is provided, including mixtures thereof for use in medical formulations.

According to a preferred embodiment of the inventive wound dressings, the antimicrobial finish on at least a portion of the surface is in the form of a coating or an integrated component of the coating composition. The antimicrobial finish preferably extends over the entire surface of the product.

In an especially preferred embodiment of the invention, the elastomer matrix has an antimicrobial impregnation.

When using the wound dressings according to the invention, PHMB is really available and capable of diffusing into the damaged body tissue.

As indicated by the preceding explanation of the inventive wound dressings, a material for producing the wound contact layer of this inventive wound dressing comprises essentially a hydrophobized active ingredient distributed in an elastomer matrix, a single phase ointment such as petroleum jelly, a hydrophobic DAB basic gel and/or a multiphase system (e.g., water-in-oil) and/or in a hydrophilic silicone gel.

Production of the inventive wound dressings is explained below. PHMB stearate can be obtained as follows: the hydrophilic PHMB hydrochloride salt is converted to a hydrophobic PHMB stearate salt as part of a metathesis reaction, forming only a harmless NaCl salt.

| | Component | Product name | Amount | |
|---|---|---|---|---|
| 1 | Stearic acid | — | 30.0 | g (105.5 mmol) |
| 2 | Sodium hydroxide | — | 3.7 | g (92.4 mmol) |
| 3 | Polyhexamethylene | Cosmocil PQ | 100.0 | g (ca. 7.7 mmol) |
| | biguanide | (20%, Arch) | | |
| | (polyhexanide) | | | |
| 4 | Water AP according | — | 300 | mL |
| | to Ph. Eur. | | | |

Procedure:

Stearic acid (Merck) is placed in 300 mL AP water, sodium hydroxide is added and the mixture is heated to approximately 80° C. Cosmocil PQ (Arch) is diluted with water (1:1), added slowly and stirred for 2 hours at approximately 80° C. The precipitate is filtered out and washed with water. The precipitate is dried overnight at approximately 40° C. and/or lyophilized.

PHMB laurate can be obtained as follows: The PHMB hydrochloride salt is converted to a PHMB stearate salt as described above. However, instead of 30.0 g stearic acid, 21.0 g lauric acid (Merck) is used.

Wound dressings according to the invention can be obtained as follows:

### Example 1

| | Amount (g) | Parts by weight (%) |
|---|---|---|
| Paraffin oil | 1120 | 71.6 |
| PHMB stearate | 56 | 3.58 |
| Copolymer | 41 | 2.62 |
| Antioxidant | 1.6 | 0.10 |
| Petroleum jelly | 125 | 7.99 |
| CMC | 221 | 14.13 |
| Total | 1564.6 | 100.0 |

The composition is prepared in a laboratory dissolver, placing 1120 g of the paraffin oil in the dissolver first and mixing with 41 g of an elastomer copolymer SEEPS (Septon 4055, Kuraray) and 1.6 g of an antioxidant (Irganox 1010) and stirred at approximately 135° C. until obtaining a homogeneous clear elastomer composition. After incorporating 125 g petroleum jelly (Vara AB, Sasol), 56 g of the PHMB stearate in powder form is added and stirred in homogeneously. Next, 221 g of the sodium carboxymethyl cellulose (CMC, Blanose 7H4XF, Aqualon) is added. The resulting elastomer composition is stirred for approximately 30 minutes more until obtaining a homogeneous composition.

The composition may also be prepared in a kneader or the like for processing of hot melt compositions in installations/equipment that is/are generally known.

The composition may be applied to the fabric (tulle) in an immersion bath at approximately 140-145° C., so that the textile fabric is completely sheathed but the interspaces/pores remain largely open to ensure the flow of exudate.

### Example 2

Like example 1, but a polymer mixture of 2% Septon 4055 and 0.6% Septon 4077 was used.

### Example 3

Like example 1, but 15% of a nonionic cellulose derivative (HPMC, Bonucel D15000) was used.

### Example 4

Like example 1, but 3.58% of a deprotonized PHMB (by adding a base, e.g., NaOH) was used instead of PHMB stearate.

### Example 5

Like examples 1 through 4, but PHMB laurate was used instead of PHMB stearate.

The invention is not limited to the exemplary embodiments described above. Instead, another idea involves the use of backing materials in the form of open mesh knits and wovens and/or polyurethane foams, such as Vivo MCF 03 (AMS). The concentration of the antimicrobial agent may be varied as a function of the use area. The deciding factor is that the antimicrobial agent has been hydrophobized. The inventive wound dressing may also contain at least one additional active ingredient, preferably one that has also been hydrophobized, such as octenidine stearate and/or metals such as silver, copper, selenium and/or metal compounds, in particular metal salts (Ag₂O, AgCl, ZnO, MgO).

Within the scope of the invention, it may be of particular importance that the wound dressings absorb only in the range of 5% to 30% of the dry weight (the aforementioned limited absorption area refers to the elastomer wound contact layer since the backing materials may lead to different results for the entire wound dressing). This limits the gelation and swelling (due to hydrocolloid solutions) so that the transport of exudate through the openings in the dressing and into the secondary dressing is not hindered.

Within the scope of the invention, the aspect that the backing has an open mesh so that it can still be assured that exudate will flow through even after the fibers forming the backing have been coated/sheathed with the elastomer composition.

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A wound dressing comprising a backing and an antimicrobially effective wound contact layer having an elastomer matrix, wherein said wound contact layer has a hydrophobized active ingredient with which the elastomer matrix is formed.

2. The wound dressing according to claim 1, wherein the hydrophobized active ingredient has a hydrophobized polymeric biguanide.

3. The wound dressing according to claim 2, wherein the hydrophobized polymer biguanide is hydrophobized polyhexamethylene biguanide (“PHMB”).

4. The wound dressing according to claim 3, wherein the hydrophobized PHMB comprises a salt of an organic acid containing 5 to 25 carbon atoms or a mixture thereof.

5. The wound dressing according to claim 1, wherein the hydrophobized-active ingredient has a melting range below 120° C.

6. The wound dressing according to claim 5, wherein the hydrophobized-active ingredient has a melting range of between 71.2 and 86.7° C.

7. The wound dressing according to claim 3, wherein the hydrophobized PHMB comprises PHMB stearate.

8. The wound dressing according to claim 1, wherein the wound contact layer comprises at least one additional hydrophobized active ingredient.

9. The wound dressing according to claim 8, wherein at least one additional hydrophobized active ingredient comprises metals or metal compounds.

10. The wound dressing according to claim 1, wherein the backing comprises a material selected from a group consisting of an open mesh knitted fabric, a woven fabric, a non-woven fabric and a polyurethane foam.

11. The wound dressing according to claim 1, wherein the elastomer matrix comprises a three-block elastomer copolymer having a polystyrene block and a polyolefin block and wherein the total polymer content is less than 3.2% by weight and is plasticized by a non-polar oil.

12. The wound dressing according to claim 11, wherein the total polymer content is 2.6% or less by weight and is plasticized by a non-polar oil.

13. The wound dressing according to claim 11, wherein the three-block elastomer copolymer comprises polystyrene-polyethylene-polybutylene-polystyrene (SEBS) or polystyrene-b-poly(ethylene/propylene)-b polystyrene (SEEPS).

14. The wound dressing according to claim 11, wherein the elastomer matrix comprises a three-block elastomer with a high molecular weight and a Brookfield viscosity of 5,000 mPas or more (for a 10% solution at 30° C.).

15. The wound dressing according to claim 14, wherein the molecular weight of the three-block elastomer is 150,000 to 600,000 daltons.

16. The wound dressing according to claim 14, wherein the molecular weight of the three-block elastomer is 200,000 to 600,000 daltons.

17. The wound dressing according to claim 14, wherein the molecular weight of the three-block elastomer is 300,000 to 600,000 daltons.

18. The wound dressing according to claim 14, wherein the molecular weight of the three-block elastomer is 400,000 to 450,000 daltons.

19. The wound dressing according to claim 11, wherein the total polymer content of the elastomer matrix comprises at least 50% of a high molecular weight elastomer with a molecular weight of approximately 150,000 to 300,000 dalton, and less than 50% of an ultrahigh molecular weight polymer with a molecular weight of approximately 400,000 to 600,000 dalton.

20. The wound dressing according to claim 11, wherein the total polymer content of the elastomer matrix comprises at least 50% of a high molecular weight elastomer with a molecular weight of approximately 200,000 to 300,000 dalton, and less than 50% of an ultrahigh molecular weight polymer with a molecular weight of approximately 400,000 to 450,000 dalton

21. The wound dressing according to claim 1, wherein the elastomer matrix contains an ionic or non-ionic hydrocolloid that is dispersed homogeneously in the matrix.

22. The wound dressing according to claim 1, wherein the active ingredient is dissolved, dispersed or distributed homogeneously in the elastomer matrix.

23. The wound dressing according to claim 1, wherein the elastomer matrix does not contain active ingredient in all areas.

24. The wound dressing according to claim 1, wherein the elastomer matrix contains a tackifier in order to increase the adhesive strength.

25. The wound dressing according to claim 1, wherein the wound contact layer has a single phase ointment having the hydrophobized active ingredient.

26. The wound dressing according to claim 23, wherein the single phase ointment having the hydrophobized active ingredient is selected from a group consisting of petroleum jelly, hydrophobized silicon gel, DAB, a multi-phase system or hydrophobic silicon gel.
